(19) United States
(12) Plant Patent Application Publication
(10) Pub. No.: US 2015/0366119 P1
(43) Pub. Date: Dec. 17, 2015

(54) Oak Tree Named Joan Lionetti

(50) ;

(71) Applicant: Nicholas Shipley, Tucson, AZ (US)

(72) Inventor: Nicholas Shipley, Tucson, AZ (US)

(21) Appl. No.: 14/120,681

(22) Filed: Jun. 17, 2014

(51) Int. Cl. A01H 5/00

(52) U.S. Cl. PLT/225

(57) ABSTRACT

A new and distinct Quercus fusiformis variety named ‘Joan Lionetti’ is characterized by dark green colored leaves, dense upreaching crown structure, excellent adaptation to conditions prevailing in the southwestern United States, with relatively low mast production.

### LATIN NAME

Quercus fusiformis

### VARIETAL DENOMINATION

‘Joan Lionetti’

### BACKGROUND OF THE INVENTION

Quercus is a large genus of the plant family Fagaceae consisting of approximately 600 species of trees and shrubs native to the Northern Hemisphere. In English, these plants are ordinarily referred to as oaks. Various oaks find usage for timber in the production of hardwood lumber, construction timbers and for cooperage. Oaks constitute important wildlife trees, providing habitat for many native animals as well as producing acorns used as food by both by humans and animals. Oaks mature into attractive, long lived trees frequently utilized in landscape plantings. The present invention relates to a new and distinct cultivar of Quercus fusiformis, an evergreen oak native from southwest Oklahoma, through Texas into northeast Mexico. Quercus fusiformis is also known as Texas Live Oak, Escarpment Live Oak and Plateau Live Oak. For many years, selected open pollinated seedlings of this species have been sold under the cultivar name ‘Heritage’ live oak. Quercus fusiformis is the most cold hardy Live Oak, tolerating temperatures down to −10 F. Texas Live Oak possesses excellent tolerance to alkaline, somewhat saline soils and to droughty conditions, resulting in this species being widely utilized in managed landscapes throughout the southwestern United States.

In 2003 the inventor grew out a lot of 5000 seedlings in Sahuarita, Ariz. From these plants, one individual possesses the unique combination of dark colored leaves, dense crown structure, upright growth form and excellent adaptation to local conditions with low mast production which is the object of this application.

### SUMMARY OF THE INVENTION

Among the features that distinguish the new Quercus fusiformis ‘Joan Lionetti’ cultivar from all other available and commercial varieties of Texas Live Oak known to the inventor are the following combination of characteristics: dark green colored leaves, dense upreaching crown structure, excellent adaptation to local conditions and relatively low mast production, resulting in lessened debris accumulation in the landscape.

Quercus fusiformis ‘Joan Lionetti’ will be used as an ornamental or shade tree well adapted to conditions found throughout the southwestern United States.

The propagation procedure for ‘Joan Lionetti’ is as follows:

Semi-hardwood cuttings are prepared (basally deleafed, about 4 inches long) and wetted in Dip & Grow™, then placed into a peat medium substrate in spring or summer in a humidity controlled mist greenhouse with internal temperature between 70 and 85 F. Cuttings root within about 6 weeks.

The foregoing characteristics and distinctions come true to form and are established and transmitted throughout succeeding propagations. The present invention has not been evaluated under all possible environmental conditions, such that the phenotype may vary with variations in environmental conditions without a change of the genotype of the plant.

### BRIEF DESCRIPTION OF THE DRAWINGS

The accompanying photographs illustrate Quercus fusiformis ‘Joan Lionetti’ growing near Tucson, Arizona, depicted in color as nearly correct as it is possible to make in a color illustration of the character.

FIG. 1 shows the 9 year old original Quercus fusiformis ‘Joan Lionetti’ growing in a cultivated area near Sahuarita, Ariz.

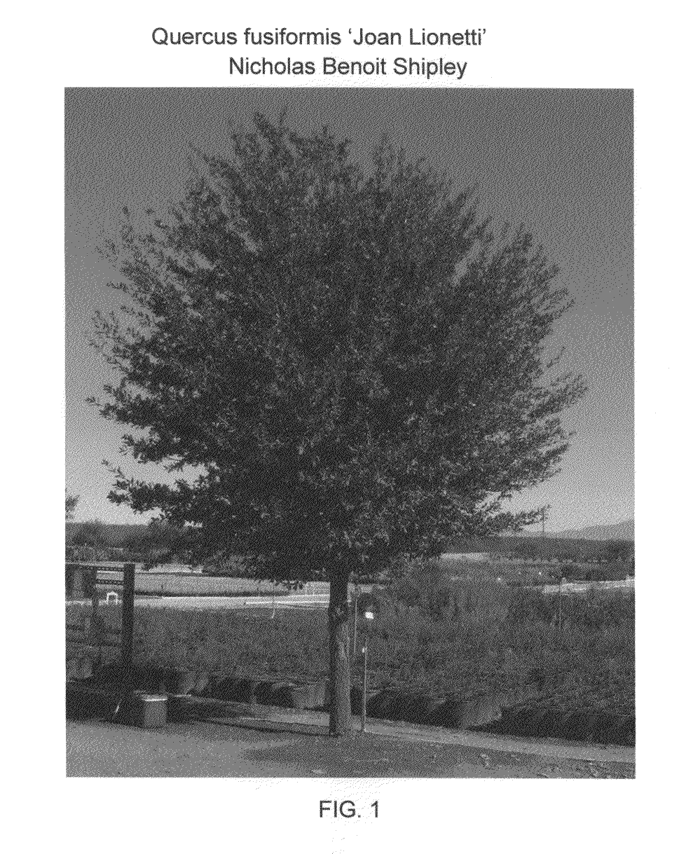

FIG. 2 is a closeup of the bark of Quercus fusiformis ‘Joan Lionetti’.

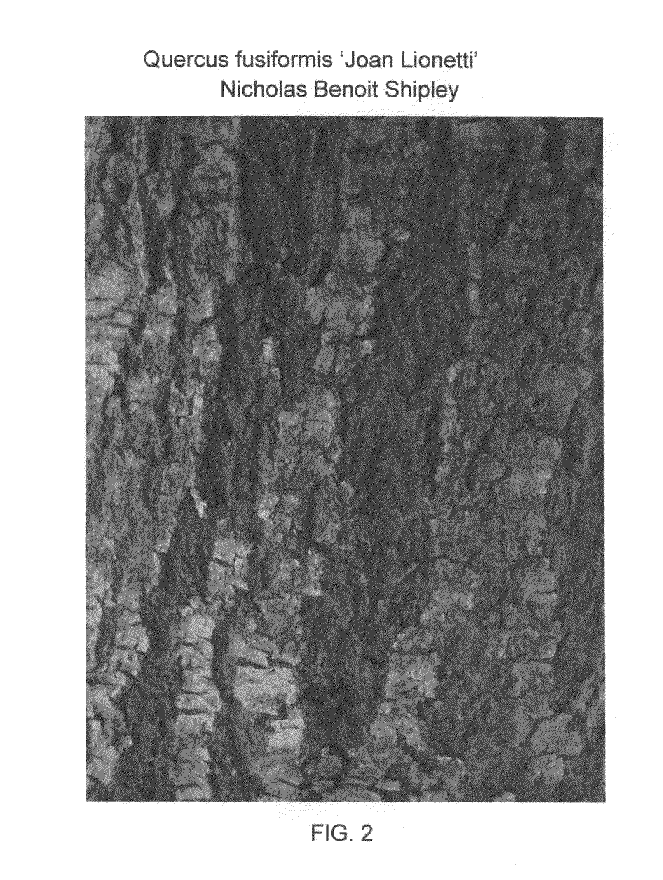

FIG. 3 is a closeup of the leaves of Quercus fusiformis ‘Joan Lionetti’

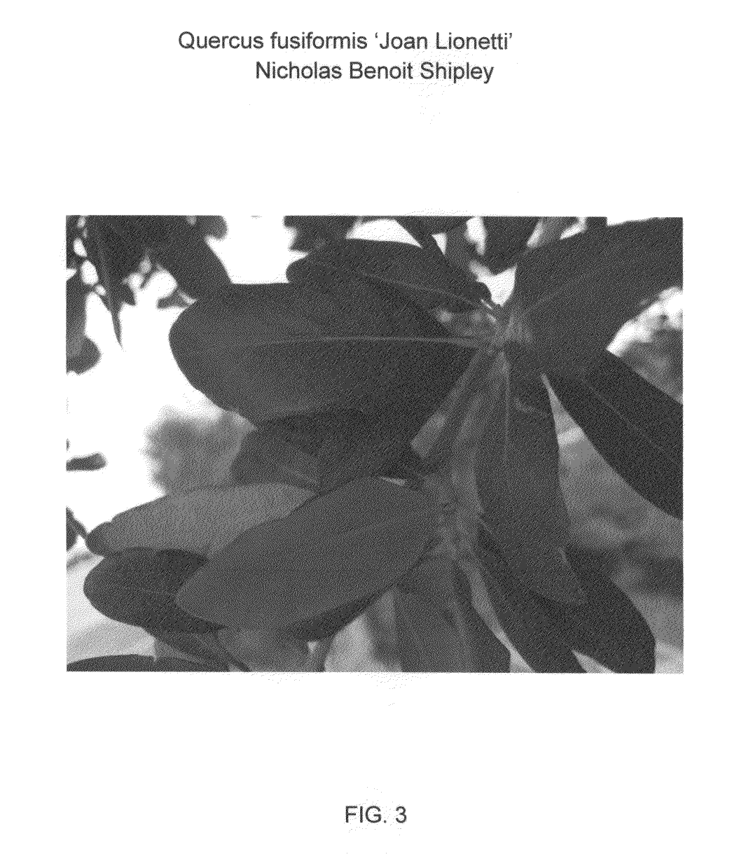

FIG. 4 is a closeup of a nearly mature acorn and associated structures of Quercus fusiformis ‘Joan Lionetti’

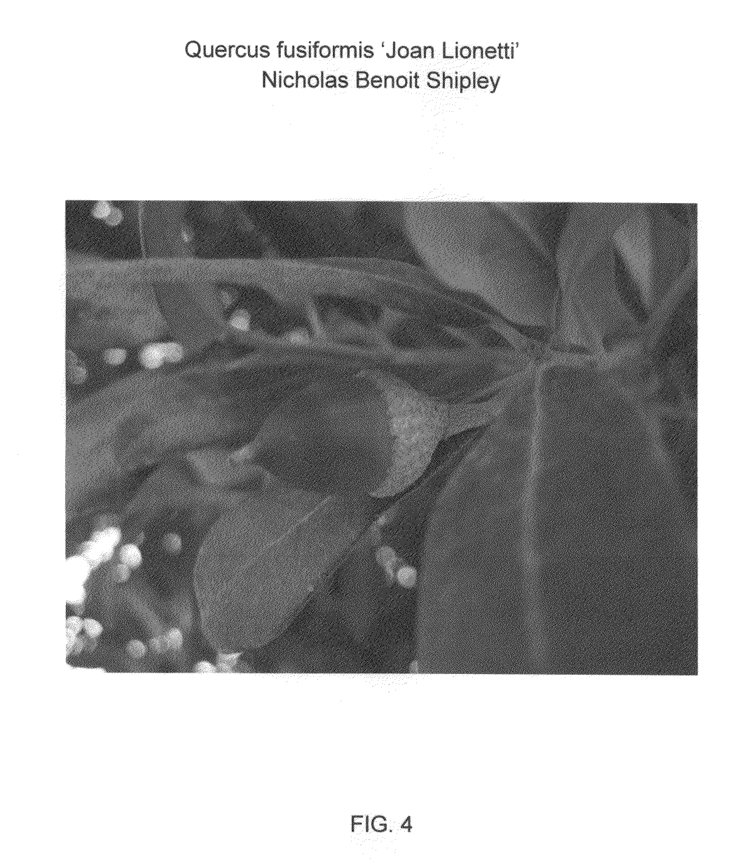

### DETAILED PLANT DESRIPTION

The following is a detailed description of the new Quercus fusiformis plant based upon measurements from a 9 year old plant growing near Sahuarita, Ariz. The color descriptions are based upon the 5^{th} edition RHS Colour Chart. Color names other than common usage are as listed in COLOR Universal Language and Dictionary of Names, by Kenneth L. Kelly and Deane B. Judd; National Bureau of Standards special publication 440. Washington, D.C. U.S. Department of Commerce, National Bureau of Standards, December 1976.
Plant name: ‘Joan Lionetti’
Species: Quercus fusiformis
Original source plants: Open pollinated seed from cultivated Quercus fusiformis
Original collection data: Seeds collected from cultivated material fall 2002
Plant size at 9 years of age: Standard tree 18 feet tall by 12 feet wide. Typical mature Texas Live Oaks generally reach around 30×30 feet at maturity. Plants grow denser, more upright and darker green in appearance than the typical range of forms normally observed in Quercus fusiformis.
Bark: fissured, varying in color from N167D to 202D to 202A
Branch angle: about 45 degrees
Dormant buds: spheroidal to oblate spheroid, imbricate, scales rounded, ciliate, nearly glabrous on surfaces, 166C.
Young, but mature stems: 197C, lanulate with small lenticels centered in longitudinal slits ⅓-⅔ mm long, stems with increasing age form an elongated reticulum, becoming rugose and glabrous.
Leaves: alternate, abaxially cupped, (length×width (mm)) 18-67×9-35, adaxial surface (147A) nearly glabrous, somewhat lustrous with scattered stellate hairs; abaxial surface (194A) densely covered with an interlocking tomentum of fine stellate hairs; petioles (198C) 4-7 mm long×1.25-1.5 mm thick, lanulate. ‘Joan Lionetti’ generally blooms in autumn, with fruits (acorns) maturing mostly in November under growing conditions typically found at Sahuarita, Ariz. Male flowers 1.5 mm, spheroidal just prior to anthesis, with 1-6 flowers/node in catkins 2.0-4.5 cm long, axis lanulate 138C, anthers about 0.5 mm, 4 (3-5) per flower, 159C, dehiscence lines 165A, barely exserted, calyx of 4-6 ovate, ciliate, but otherwise glabrous sepals (N144C) formed into a cup, pollen 4C; female flowers generally solitary, axillary, spheroidal, 5 mm long×2.5 mm broad; peduncle 4-5 mm long×1.5 mm thick, terete, lanate; involucre 2.5 mm broad, visually 4 ranked at flowering, color 1990; bracts lanulate, ciliate, rounded in shape, color 199C; ovary ovoid, mucronate, color 137C.
Acorn (fruit): ovoid, acuminoid 27 mm long×14 mm in diameter, basally N199D, distally 200A-C, surface slightly glaucous, shiny when rubbed, obscurely striate, but noticeably more striate than typical Quercus fusiformis, with persistent stigma, fruit basal scar raised 161D, low hemispherical, 3 mm in diameter, with 20-21 vascular bundles; involucre 8-9 ranked 7-8 mm long ×11-12 mm in diameter, obconical, bracts lanulate, ciliate, basal bracts rounded-ovate, distal bracts ovate-acuminate, bract color 199C.

### COMPARISONS TO RELATED QUERCUS

Compared to the only other commonly available Texas Live Oak, the open pollinated ‘Heritage’ oak, ‘Joan Lionetti’ is consistent with its dense, upright form, dark green color and excellent adaptability. ‘Heritage’ oak, while selected for plant form, is inconsistent in growth form and characteristics, color and adaptability, and a portion of all the trees in a planting commonly fail over a period of years. ‘Joan Lionetti’ oak is expected to provide a more consistent tree in the landscape due to its clonal nature.

I claim:

1. A new and distinct Quercus fusiformis plant substantially as described and illustrated herein.
